(19) United States
(12) Patent Application Publication
(10) Pub. No.: US 2025/0332074 A1
(43) Pub. Date: Oct. 30, 2025

(54) WATER-IN-OIL EMULSION COMPOSITION

(71) Applicant: L'OREAL, PARIS (FR)

(72) Inventor: Sonal PATEL, Iselin, NJ (US)

(73) Assignee: L'OREAL, PARIS (FR)

(21) Appl. No.: 18/651,001

(22) Filed: Apr. 30, 2024

(51) Int. Cl. A61K 8/06; A61K 8/25; A61K 8/37; A61K 8/60; A61K 8/81; A61Q 1/02

(52) U.S. Cl. CPC A61K 8/064; A61K 8/25; A61K 8/37; A61K 8/602; A61K 8/8111; A61Q 1/02

(57) ABSTRACT

The present invention relates to a composition in the form of a water-in-oil emulsion, including, (a) one or more esters formed from at least one fatty acid having more than 13 carbon atoms; (b) a polymer component including one or more polymerized (iso)butenes; (c) at least about 3% by weight of one or more carbohydrate fatty acid ester emulsifiers; (d) one or more fillers comprising hydrophobic silica; and (e) one or more pigments. The composition is substantially free of silicones & alkanes.

### TECHNICAL FIELD

The present invention relates to the field of personal care and cosmetic products, particularly, to a makeup composition in the form of water-in-oil emulsions. The present invention further relates to a method for making up and caring of keratin materials and preferably concealing imperfections on the skin.

### BACKGROUND

Many consumers desire to use cosmetic and personal care compositions that enhance the appearance of keratin materials and especially the skin, in particular the optical cosmetic effect of the skin, such as brightening effect, coverage effect and radiance effect of the skin.

The skin is not a smooth surface of uniform color, and has reliefs or at least microreliefs such as pores, wrinkles, fine lines, dark spots, scars, blemishes, black eyes and dry areas, which form a somewhat irregularity on the surface of the face. Further, the complexion is not always satisfactory, e.g., a dull appearance. Cosmetic compositions, for example foundations and concealers, are commonly used to give the skin an esthetic color, and also to enhance the beauty of irregular skin, by making it possible to hide marks, and dyschromia, to reduce the visibility of relief imperfections, such as pores and wrinkles, and to conceal dark spots and acne marks.

In general, a concealer has better covering power than a foundation, is more attached to skin, and can mask the flaws such as dark spots, scars, blemishes, black eyes, so that the color of the surface of human skin are more uniform. Concealers are either too thin or too dense to make the makeup look unnatural, and when used, many consumers therefore desire to obtain a natural finish full coverage, long wearing, feels fresh, light weight along with smooth super creamy texture, good tack, good skin adhesion & pleasant gliding sensation at application. A large variety of cosmetics have already been investigated in this regard, but it is still a technical difficulty to achieve desirable concealing coverage in one stable formulation. Therefore, there is a need for a composition, e.g., as a concealer, which can provide one or more benefits including a natural finish full coverage effect, desirable freshness and is stable over a long period of time and a wide temperature range for storage and use.

### BRIEF SUMMARY

In one aspect of the present invention, there is provided a cosmetic composition suitable for making up and caring of the skin, the composition including (a) one or more esters formed from at least one fatty acid having more than 13 carbon atoms; (b) a polymer component comprising one or more polymerized (iso)butenes; (c) at least about 3% by weight of one or more a carbohydrate fatty acid ester emulsifier; (d) one or more fillers comprising hydrophobic silica; and (e) one or more pigments. The composition is substantially free of silicones and alkanes.

According to certain embodiments, one or more esters formed from at least one fatty acid having more than 13 carbon atoms may include esters of myristic acid, esters of palmitic acid, esters of stearic acid and mixtures thereof. The polymer component comprising one or more polymerized (iso)butenes may include polyisobutene, hydrogenated polyisobutene, and mixtures thereof. One or more carbohydrate fatty acid ester emulsifier may include sorbitan palmitate, sorbitan isostearate, sorbitan trioleate and mixtures thereof. One or more fillers comprising hydrophobic silica may include hydrophobically treated pyrogenic silica, hydrophobic silica aerogel, and mixtures thereof. One or more pigments may include mineral pigments, organic pigments, pearlescent pigments, and mixtures thereof.

In certain embodiments, one or more fillers comprising hydrophobic silica and the polymer component comprising polymerized (iso)butene are present in a weight ratio of hydrophobic silica to polymerized (iso)butene that is from about 1:50 to about 1:10.

The cosmetic composition, may, in yet other embodiments meet one, a plurality, or even all of these above-described limitations.

In another aspect of the present invention, there is provided a method for making up and caring of keratin materials, preferably the skin, comprising applying to said skin, the any of the above-described cosmetic compositions.

These and other features, aspects, and advantages of the present invention will be better understood with reference to the following description and claims.

### DETAILED DESCRIPTION

Those skilled in the art will be aware that the present invention is subject to variations and modifications other than those specifically described. It is to be understood that the present invention includes all such variations and modifications. The invention also includes all such, features, compositions, and compounds referred to or indicated in this specification, individually or collectively, and any and all combinations of any or more of such steps or features. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions.

For convenience, before further description of the present invention, certain terms employed in the specification, and examples are described here. These definitions should be read in light of the remainder of the invention and understood by a person of skill in the art. The terms used herein have the meanings recognized and known to those of skill in the art, however, for convenience and completeness, particular terms and their meanings are set forth below.

As used herein, the expression “at least one” is synonymous with “one or more” and thus includes individual components as well as mixtures/combinations.

“A” or “an” as used herein means “at least one.”

The terms “comprise,” and “include” and related forms of these words are used in the inclusive, open sense, meaning that additional elements (or steps) may be included. It is not intended to be construed as “consists of only” or to exclude any other elements or steps.

Further, the term “including” is used to mean “including but not limited to”. “including” and “including but not limited to” are used interchangeably.

The term “substantially free of (a component)” as used herein, refers to the systems or compositions contain no appreciable amount of the component, for example, no more than about 1% by weight, or no more than about 0.5% by weight, or no more than about 0.3% by weight, such as no more than about 0.1% by weight, based on the weight of the system or composition according to embodiments of the invention.

The term “free” or “completely free of (a component)” as used herein, refers to the systems or compositions do not contain the component in any measurable degree by standard means.

The term “INCI” is an abbreviation of International Nomenclature of Cosmetic Ingredients, which is a system of names provided by the International Nomenclature Committee of the Personal Care Products Council to identify cosmetic or personal care ingredients.

The term “anhydrous”, as used herein, refers to a composition comprising less than 5% by weight of water, or even less than 2% by weight of water, or even less than 1% by weight of water relative to the total weight of the composition, or even is free of water.

The term “keratin material” as used herein refers to hair, skin, eyebrow, lips, and nails and more particularly the skin of the areas such as the face, the checks, the hands, the body, the legs and thighs, the area around the eyes, and the eyelids.

The term “colorant”, as used herein, refers to its generally accepted meaning in the art. It refers to a compound that is capable of producing a colored optical effect when it is formulated in a sufficient amount in a suitable cosmetic medium.

The term “Polymer” as used herein, refers to a compound which is made up of at least two monomers.

The term “room temperature” as used herein refers to 25° C.

The term “Actives basis” as used herein, refers to only the particular component of ingredient (e.g., in a composition) and ignoring other chemically unrelated components that may also be present in the same raw material source of that particular component.

All percentages, parts and ratios are based upon the total weight of the compositions of the present invention unless otherwise indicated. Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the ratio. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A: B ratio of 1:2.5. “About 1:10,” means from 1:8.5 to 1:11.5, preferably 1:9 to 1:11. As one skilled in the art will understand, the modifier “about” also encompasses the exact number or ratio specified. Unless otherwise indicated, all concentrations shown as percentages are concentrations by weight and also, unless otherwise indicated, relate to the entire composition as a whole.

Numerical ranges, unless specifically stated otherwise, are inclusive of endpoints and meant to include all combinations and sub-combinations. Notably, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. Further as used herein a range of ratios is meant to include every specific ratio within, and combination of subranges between the given ranges.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the invention, the preferred methods and materials are now described.

The present invention is not to be limited in scope by the specific embodiments described herein, which are intended for the purposes of exemplification only. Functionally equivalent products, compositions, and methods are clearly within the scope of the invention, as described herein.

Embodiments herein provide a cosmetic composition in the form of water-in-oil emulsion, for making up and caring of the skin. The disclosed embodiments include a method for making up and caring of the skin. In various embodiments herein, the composition is a liquid concealer composition having improved cosmetic properties. The composition, according to embodiments herein, has good stability with the desired smooth creamy texture, full coverage and enhanced ease of application. Further, the composition, according to embodiments herein, gives the skin an esthetic color, and also to enhance the beauty of irregular skin, by making it possible to mask the flaws such as dark spots, scars, acne marks, blemishes, black eyes and dyschromia. The embodiments herein, further, include a method for the preparation of the cosmetic composition.

### Cosmetic Composition

Thes present invention relates to a cosmetic composition for making up and caring of the skin. In certain embodiments, the composition provides light weight, smooth creamy texture, feels fresh make up look. Further in certain embodiments, the cosmetic composition offers enhanced benefits such as full coverage, improved hydration with radiant finish.

In certain embodiments, the composition is in the form of a water-in-oil emulsion. The term “water-in-oil emulsion” as used herein refers to a composition comprising a continuous oily phase and an aqueous phase, which are immiscible; the aqueous phase being dispersed in the form of droplets in the continuous oily phase so as to obtain a macroscopically homogeneous composition.

In certain embodiments, said composition may include (a) one or more esters formed from at least one fatty acid having more than 13 carbon atoms; (b) polymer component comprising one or more polymerized (iso)butenes; (c) at least about 3% by weight of one or more carbohydrate fatty acid ester emulsifier; (d) one or more fillers comprising hydrophobic silica and (e) one or more pigments. wherein the composition is substantially free of silicones and alkanes.

The composition, according to embodiments herein, is a liquid water-in oil emulsion cosmetic composition.

### Ester of a >C₁₃ Fatty Acid

The composition, according to embodiments herein, includes one or more esters of >C₁₃ fatty acid(s). By “ester of a >C₁₃ fatty acid,” it is meant that the ester is formed from one or more fatty acids having more than 13 carbon atoms, such as more than 14 carbon atoms, such as from 15 carbon atoms to 20 carbon atoms, such as from 15 to 18 carbon atoms.

The composition, according to embodiments herein, includes one or more esters of >C₁₃ fatty acid(s) formed by reacting one or more fatty acids having more than 13 carbon atoms with one or more alcohols.

According to certain preferred embodiments, the ester of a >C₁₃ fatty acid is formed from a one or more fatty acids chosen from palmitic acid, stearic acid, myristic acid, arachidic acid, behenic acid, myristoleic acid, palmitoleic acid, sapienic acid, oleic acid linoleic acid, and combinations thereofFurther, the alcohol(s) from which the ester of a >C₁₃ fatty acid is formed may be saturated or unsaturated, linear or branched aliphatic monoalcohols or polyalcohols.

Preferably, for esters of monoalcohols, at least one among the alcohol and the acid from which the esters of the present invention are derived is branched.

Among the monoesters of monoacids and of monoalcohols, mention may be made of alkyl myristates such as ethyl myristate, ethylhexyl myristate or isopropyl myristate, alkyl palmitates such as ethyl palmitate, ethylhexyl palmitate or isopropyl palmitate, alkyl stearates such as ethyl stearate, ethylhexyl stearate or isopropyl stearate, and isocetyl stearate.

According to certain embodiments, the ester of a >C₁₃ fatty acid is an ester “oil,” meaning that the ester of a >C₁₃ fatty acid is liquid at ambient temperature (20° C.) and standard pressure.

According to certain preferred embodiments, the ester of >C13 fatty acid may be chosen from ethylhexyl myristate, ethylhexyl palmitate, ethylhexyl stearate or mixtures thereof.

In various embodiment, the amount of one or more esters formed from at least one fatty acid having more than 13 carbon atoms in the composition, may be present in a weight range of about 5% to about 20%, preferably in a weight range of about 8% to about 15% relative to the total weight of the composition.

For example, in various embodiments, the amount of one or more esters formed from at least one fatty acid having more than 13 carbon atoms in the composition may range from about 6% to about 18%, from about 6% to about 16%, from about 6% to about 14%, from about 6% to about 12%, from about 6% to about 10%, from about 7% to about 18%, from about 7% to about 16%, from about 7% to about 14%, from about 7% to about 12%, from about 7% to about 10%, from about 8% to about 18%, from about 8% to about 16%, from about 8% to about 14% or from about 8% to about 12% relative to the total weight of the composition.

For example, one or more esters formed from at least one fatty acid having more than 13 carbon atoms in the composition may be about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the composition, including any range using any of the foregoing as upper and lower limits.

### Polymer Component including Polymerized (Iso)Butene

The composition may also include a polymer component. Polymer component may consist of one or more polymers that function to provide film formation, viscosity modification, skin conditioning and water resistance. The polymer component, according to embodiments herein, may include a polymerized butene preferably polymerized (iso)butene. The polymerized (iso)butene may be chosen from polyisobutene, hydrogenated polyisobutene, or mixtures thereof.

In an embodiment, the polymer component may include a hydrogenated polyisobutene such as polymer having the INCI designation, Hydrogenated polyisobutene.

In an embodiment, the hydrogenated polyisobutene or isoparaffin may be prepared by the co-polymerization of isobutene and n-butene, followed by hydrogenation. Hydrogenated polyisobutene denotes a branched hydrocarbon which comprises at least one side chain such as a methyl group, which may be represented by the following chemical formula:

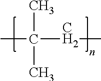

In another embodiment, Hydrogenated polyisobutene may be referred to as isoparaffin which may be categorized as light liquid isoparaffin, liquid isoparaffin, and heavy liquid isoparaffin.

Light liquid isoparaffin may have a repeating unit of —(C(CH3)2—CH2)— with a polymerization degree of 4 to 6, and may have a viscosity of from 1 to less than 10 mm2/s at 20° C. Light liquid isoparaffin may be volatile. As an example of liquid paraffin, mention may be made of Parleam® 4 (INCI name: Hydrogenated polyisobutene) by NOF Corp.

Liquid isoparaffin which may have a repeating unit of —(C(CH3)2—CH2)— with a polymerization degree of 5 to 10, and may have a viscosity of from 10 to 100 mm2/s at 20° C. As an example of liquid paraffin, mention may be made of Parleam® (INCI name: Hydrogenated polyisobutene) and Parleam® Lite (INCI name: Hydrogenated polyisobutene) by NOF Corp.

Heavy liquid isoparaffin which may have a repeating unit of —(C(CH3)2—CH2) — with a higher polymerization degree, and may have a viscosity of more than 100 mm2/s at 20° C. As an example of liquid paraffin, mention may be made of Parleam® V (INCI name: Hydrogenated polyisobutene), Parleam® HV (INCI name: Hydrogenated polyisobutene), and Parleam® SV (INCI name: Hydrogenated polyisobutene) by NOF Corp.

In an embodiment, polymerized (iso)butene is chosen from light liquid isoparaffin, liquid isoparaffin, and heavy liquid isoparaffin or mixtures thereof.

In a preferable embodiment, the polymer component including polymerized (iso)butene is liquid isoparaffin, for example such as those sold under the reference Parleam® (INCI name: Hydrogenated polyisobutene) by NOF Corp.

In various embodiment, the amount of a polymer component including polymerized (iso)butene in the composition may be present in a weight range of about 5% to about 20%, preferably in a weight range of about 8% to about 12% relative to the total weight of the composition.

For example, in various embodiments, a polymer component including polymerized (iso)butene in the composition may range from about 6% to about 18%, from about 6% to about 16%, from about 6% to about 14%, from about 6% to about 12%, from about 6% to about 10%, from about 7% to about 18%, from about 7% to about 16%, from about 7% to about 14%, from about 7% to about 12%, from about 7% to about 10%, from about 8% to about 18%, from about 8% to about 16%, from about 8% to about 14% or from about 8% to about 12% relative to the total weight of the composition.

For example, one or more dimethicones having a viscosity less than about 3 centistokes in the composition may be about 5%, about 6%, about 7%, about 8%, about 9%, about 10%, about 11%, about 12%, about 13%, about 14%, about 15%, about 16%, about 17%, about 18%, about 19%, or about 20% by weight, relative to the total weight of the composition, including any range using any of the foregoing as upper and lower limits.

### Carbohydrate Fatty Acid Ester Emulsifier

The composition, according to embodiments herein, may include one or more carbohydrate fatty acid ester emulsifiers may be chosen from esters of saccharide polyol with fatty acids. As one skilled in the art will recognize, the carbohydrate fatty acid ester emulsifier is generally an amphiphilic compound containing one or more hydrophilic moieties linked to hydrophobic aglycone. In an embodiment, the carbohydrate fatty acid ester emulsifier may include esters of polyols with fatty acids.

In a preferred embodiment, the fatty acid may be saturated or unsaturated, preferably saturated and linear or branched, preferably branched. More preferably, the fatty acid is saturated and branched. In another preferred embodiment of the present invention, the saccharide polyol of the carbohydrate fatty acid ester emulsifier comprises at least one sugar alcohol. The sugar alcohol may be chosen from mannitol, erythritol, xylitol, sorbitol, arabitol, pentaerythritol, and mixtures thereof. Preferably, the sugar alcohol is sorbitol.

In a further preferred embodiment of the present invention, one or more carbohydrate fatty acid ester emulsifiers may be chosen from sorbitol esters of a C₈-C₂₄, preferably C₁₂-C₂₂, fatty acid. More preferably, one or more carbohydrate fatty acid ester emulsifiers may be chosen from sorbitol esters of a saturated and branched C₈-C₂₄, preferably C₁₂-C₂₂, fatty acid or acids. In an embodiment, the sorbitol esters of C₈-C₂₄, and polyoxyalkylenated derivatives thereof may be chosen from sorbitan palmitate, sorbitan isostearate, sorbitan trioleate and esters of fatty acids.

In an embodiment, one or more carbohydrate fatty acid ester emulsifiers may be chosen from sorbitan palmitate, sorbitan isostearate, sorbitan trioleate and esters of fatty acids. In a preferable embodiment, one or more carbohydrate fatty acid ester emulsifier may include sorbitan isostearate.

In various embodiments, the amount of one or more carbohydrate fatty acid ester emulsifiers in the composition may be present in a weight range of about 0.05% to about 8%, preferably in a weight range of about 3% to about 7% relative to the total weight of the composition.

For example, in various embodiments, one or more carbohydrate fatty acid ester emulsifiers in the composition may range from about 1% to about 8%, from about 1% to about 7%, from about 1% to about 6%, from about 1% to about 5%, from about 1% to about 4%, from about 2% to about 8%, from about 2% to about 7%, from about 2% to about 6%, from about 2% to about 4%, from about 3% to about 8%, from about 3% to about 7%, from about 3% to about 6%, from about 3% to about 4%, from about 4% to about 8%, from about 4% to about 7%, from about 4% to about 6%, from about 5% to about 8%, from about 5% to about 7%, from about 5% to about 6%, from about 6% to about 8%, from about 6% to about 7% relative to the total weight of the composition.

For example, one or more carbohydrate fatty acid ester emulsifiers in the composition may be about 1%, about 2%, about 3%, about 4%, about 5%, about 6%, about 7%, or about 8%, by weight, relative to the total weight of the composition, including any range using any of the foregoing as upper and lower limits.

In certain embodiments, one or more carbohydrate fatty acid ester emulsifiers in the composition may be at least about 3%, at least about 4%, at least about 5%, at least about 6%, at least about 7% or at least about 8% by weight, relative to the total weight of the composition.

### Fillers

The composition, according to embodiments herein, may include one or more fillers. The one or more fillers includes hydrophobic silica and optionally, additional fillers. By “filler” it is meant a particulate material, exclusive of pigments (described below), generally dispersible in the water phase or the oil phase of the cosmetic composition.

### Silica

The composition, according to embodiments herein, include one or more hydrophobic silicas. The silica, according to embodiments herein also known as silicone dioxide. As one skilled in the art will recognize, the silica is generally a crystal that is colorless or a white powder that is tasteless and odorless which has a lot of different functions in cosmetics. It can act as an absorbing agent, bulking agent, anti-caking agent, and as abrasive.

The hydrophobic silica, according to embodiments herein, may be chosen from hydrophobic pyrogenic silica and hydrophobic silica aerogel or mixture thereof.

According to a preferred embodiment, the composition may include hydrophobic silica aerogel. The hydrophobic silica aerogel particles used in the present invention may have a specific surface area per unit mass (SM) ranging from 600 to 800 m²/g and a size expressed as mean diameter in volume (D[0.5] ) ranging from 5 to 20 μm and or more preferably from 5 to 15 μm.

The term “hydrophobic silica”, as used herein, refers to any silica whose surface is treated with silylation agents, for example with halogenated silanes such as alkylchlorosilanes, siloxanes, in particular dimethylsiloxanes such as hexamethyl disiloxane, or silazanes, so as to functionalize the OH groups with Si—Rₙ silyl groups, for example, trimethylsilyl groups.

The hydrophobic silica aerogels, according to embodiments herein, include for example the aerogel marketed under the name VM-2260 (INCI name: Silica silylate), by the company Dow Corning, whose particles have an average size of about 1000 microns and a specific surface per unit mass ranging from 600 to 800 m²/g .

The aerogel marketed under the name VM-2270 (INCI name: Silica silylate), by the company Dow Corning, is preferably used in various embodiments herein, the particles of which have an average size ranging from 5-15 microns and a specific surface area per unit of mass ranging from 600 to 800 m²/g .

The aerogel marketed under the name Aerogel TLD 201® (INCI name: Silica silylate), by the company Cabot.

In various embodiment, hydrophobic silica may be present in a weight range of about 0.05% to about 2.5%, preferably in a weight range of about 0.2% to about 1% relative to the total weight of the composition.

For example, in various embodiments, hydrophobic silica in the composition may range from about 0.1% to about 2%, from about 0.1% to about 1.5%, from about 0.1% to about 1%, from about 0.1% to about 0.8%, from about 0.1% to about 0.7%, from about 0.1% to about 0.5%, from about 0.1% to about 0.6%, from about 0.2% to about 2%, from about 0.2% to about 1.5%, from about 0.2% to about 1%, from about 0.2% to about 0.8%, from about 0.2% to about 0.7%, from about 0.2% to about 0.6%, from about 0.2% to about 0.5%, from about 0.3% to about 2%, from about 0.3% to about 1.5%, from about 0.3% to about 1%, from about 0.3% to about 0.8%, from about 0.3% to about 0.7%, from about 0.3% to about 0.6%, or from about 0.3% to about 0.5% relative to the total weight of the composition.

For example, in various embodiments, hydrophobic silica in the composition may be about 0.05%, about 0.1%, about 0.2%, about 0.3%, about 0.4%, about 0.5%, about 0.6%, about 0.7%, about 0.8%, about 1%, about 1.5% or about 2% by weight, relative to the total weight of the composition, including any range using any of the foregoing as upper and lower limits.

### Clays

The composition, according to embodiments herein, may include optionally additional fillers for example clays. As one skilled in the art will recognize, the clay is generally naturally occurring mineral based substance. The clay, according to embodiments herein, may include natural and/or synthetic clays.

Clays are silicates containing a cation that can be chosen from calcium, magnesium, aluminum, sodium, potassium, lithium cations, or mixtures thereof. Examples of clays include clays of smectite family, such as montmorillonites, hectorites, bentonites, beidellites and saponites; vermiculite family, such as stevensite, chlorites, etc. Such clays can be of natural or synthetic origin.

Embodiments herein may also include organophilic clays. Organophilic clays are clays modified with a chemical compound chosen from among quaternary amines, tertiary amines, amino acetates, imidazolines, amino soaps, fatty sulphates, alkyl aryl sulphonates, amine oxides, and mixtures thereof.

In some embodiments, the hectorites may be modified by a quaternary compound, more precisely by a halide, such as a chloride, of ammonium fatty acid C10 to C22, such as hectorite modified by chloride di-stearyl di methyl ammonium (CTFA name: Disteardimonium hectorite), such as, for example, that marketed under the name Bentone 38V, Bentone 38V CG, Bentone EW CE, by the company ELEMENTIS; stearalkonium Hectorites such as Bentone 27V, disteardimonium hectorites such as Bentone Gel ISD V, marketed by the company Elementis. Examples of bentonites includes quaternium-18 bentonites such as those sold under the names Bentone 34, marketed by the company Elementis, Claytone 40, Tixogel VP by the company United catalyst by the company Southern Clay, stearalkonium bentonites such as those sold under the names Tixogel LG by the company United Catalyst, Claytone AF, Claytone APA by the company Southern Clay; quaternium-18/benzalkonium bentonite such as those sold under the name Claytone HT by the company Southern Clay. In a preferable embodiment, the clays are chosen in particular from hectorites modified with a C₁₀ to C₂₂ ammonium halide, in particular chloride, such as hectorite modified with di-stearyl di-methyl ammonium chloride, or alternatively bentone gels, in particular, disteardimonium hectorite in isododecane. In an embodiment, the filler is disteardimonium hectorite. In another embodiment, the filler is disteardimonium hectorite in isododecane.

In some embodiment, the optional additional filler, is chosen from hydrophobic hectorite and hydrophobic bentonite or mixture thereof.

In various embodiment, the amount of total filler including hydrophobic silica and optionally additional fillers may be present in a range of about 0.05% to about 2.5%, preferably in a weight range of about 0.1% to about 2% relative to the total weight of the composition.

For example, in various embodiments, the amount of total filler including hydrophobic silica and optionally additional fillers may range from about 0.1% to about 2%, from about 0.1% to about 1.5%, from about 0.1% to about 1%, from about 0.1% to about 0.8%, from about 0.1% to about 0.7%, from about 0.1% to about 0.6%, from about 0.1% to about 0.5%, from about 0.2% to about 2%, from about 0.2% to about 1.5%, from about 0.2% to about 1%, from about 0.2% to about 0.8%, from about 0.2% to about 0.7%, from about 0.2% to about 0.6%, from about 0.2% to about 0.5%, from about 0.3% to about 2%, from about 0.3% to about 1.5%, from about 0.3% to about 1%, from about 0.3% to about 0.8%, from about 0.3% to about 0.7%, from about 0.3% to about 0.6%, or from about 0.3% to about 0.5% relative to the total weight of the composition.

For example, in various embodiments, the amount of total filler including hydrophobic silica and optionally additional fillers may be about 0.05%, about 0.1%, about 0.2%, about 0.3%, about 0.4%, about 0.5%, about 0.6%, about 0.7%, about 0.8%, about 1%, about 1.5% or about 2% by weight, relative to the total weight of the composition, including any range using any of the foregoing as upper and lower limits.

In some embodiments, the filler including hydrophobic silica, preferably hydrophobic silica aerogel of the present invention may be present in a range of about 0.1%, 0.2% or 0.3% to about 0.5% 0.6%. 0.7%, 0.8%, 1%, 1.5% or 2.0% by weight, more preferably in a range of about 0.2% to about 1.0% by weight relative to the total weight of the composition, including all ranges and subranges in between. In some embodiments, the polymer component including polymerized (iso)butene preferably is hydrogenated polyisobutene of the present invention may be present in a range of about 5.0%, 6.0%, 7.0%, 8.0% or 9.0% to about 10%, 11%, 12%, 13%, 14%, 15%, 16%, 17%, 18% by weight, more preferably in a range of about 8.0% to about 12.0% by weight relative to the total weight of the composition, including all ranges and subranges in between. In some embodiments, the hydrophobic silica aerogel and hydrogenated polyisobutene may be present in the composition in a weight ratio that is from about 1:50, 1:40, 1:30, 1:20, 1:10 to about 1:10, 1:20, 1:30, 1:40, 1:50.

### Colorants

The composition, according to embodiments herein, may include one or more colorant. By “Colorant” it is meant a substance used for coloring material. The colorant may in particular be selected from pigments and dyes, or mixtures thereof.

The term “pigments” is used to mean white or colored, mineral or organic particles, insoluble in an aqueous medium, intended to color and/or opacify the resulting composition and/or deposit. These pigments may be white or colored, mineral and/or organic.

The pigments may be chosen from mineral pigments, organic pigments, pearlescent pigments, or mixtures thereof.

In an embodiment of the present invention, the pigment is chosen from mineral pigments.

The term “mineral pigment” is used to mean any pigment which meets the definition of the Ullmann encyclopedia in the chapter on inorganic pigments. Examples of mineral pigments suitable for use in the present disclosure include zirconium or cerium oxides, as well as zinc, iron (black, yellow, or red) or chromium oxides, manganese violet, ultramarine blue, chromium hydrate and ferric blue, titanium dioxide, metal powders such as aluminum powder and copper powder. The mineral pigments, such as Ta₂O₅, Ti₃O₅, Ti₂O₃, TiO, ZrO₂ mixed with TiO₂, ZrO₂, Nb₂O₅, CeO₂, ZnS can also be used in the composition of the present invention.

The size of the pigment suitable for use in the present invention is generally in a range of 0.1 μm to 10 μm, preferably in a range of 0.2 μm to 5 μm, more preferably in a range of 0.3 μm to 1μm.

In an embodiment of the present invention, the pigments have a size characterized by a D[50] in a range of 0.1 μm to 10 μm, preferably in a range of 0.2 μm to 5 μm, more preferably in a range of 0.3 μm to 1 μm. D[50] refers to the median particle size and represents the maximum size that 50% by volume of the particles present.

In an embodiment of the present invention, the mineral pigment comprises a lipophilic or hydrophobic coating.

In one more embodiment of the present invention, the pigments may be coated with at least one compound chosen from metal soaps; N-acylated amino acids or salts thereof; lecithin and derivatives thereof; isopropyl trisostearyl titanate; isostearyl sebacate; natural vegetable or animal waxes; polar synthetic waxes; fatty esters; phospholipids; silicone surfactants such as organopolysiloxanes, alkylalkoxysilanes such as triethoxycaprylylsilane; fluorinated surfactants such as perfluoroalkyl phosphates, perfluoropolyethers, polytetrafluoropolyethylenes (PTFE), perfluoroalkanes, perfluoroalkyl silazanes, hexafluoropropylene polyoxides, polyorganosiloxanes comprising perfluoroalkyl perfluoropolyether groups; fluorosilicone surfactants such as perfluoroalkyl dimethicones, perfluoroalkyl silanes and perfluoroalkyl trialkoxysilanes; and mixtures thereof.

In another embodiment of the present invention, the pigments may be coated with an N-acylated amino acid or a salt thereof, which may comprise an acyl group having 8 to 22 carbon atoms, such as a 2-ethyl hexanoyl, caproyl, lauroyl, myristoyl, palmitoyl, stearoyl, cocoyl group. The amino acid can be for example lysine, glutamic acid, or alanine. The salts of these compounds may be aluminum, magnesium, calcium, zirconium, zinc, sodium, potassium salts.

In yet another embodiment of the present invention, the pigments may be coated with an N-acylated amino acid derivative, preferably a glutamic acid derivative and/or a salt thereof, more preferably a stearoyl glutamate, such as aluminum stearoyl glutamate.

Examples of pigments treated with aluminum stearoyl glutamate, include the titanium dioxide pigments and the black, red, and yellow iron oxide pigments sold under the trade name NAI® by the company MIYOSHI KASEI.

According to embodiments herein, the pigments can be coated with isopropyl triisostearyl titanate. As examples of pigments treated with isopropyl titanium triisostearate (ITT), mention may be made of those sold under the trade reference BWBO-12® (iron Oxide CI77499 and isopropyl titanium triisostearate), BWYO-12® (iron oxide CI77492 and isopropyl Titanium triisostearate) and BWRO-12® (iron oxide CI77491 and isopropyl titanium triisostearate) by the KOBO company.

The pigments that can be used according to the present invention can also be organic pigments.

The organic pigment may in particular be selected from nitroso, nitro, azo, xanthene, quinoline, anthraquinone, phthalocyanine, metal complex compounds, isoindolinone, isoindoline, quinacridone, perinone, perylene, diketopyrrolopyrrole, thioindigo, dioxazine, triphenylmethane, or quinophthalone.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100, 74160, the yellow pigments coded in the Color Index under the references CI 11680, 11710, 15985, 19140, 20040, 21100, 21108, 47000, 47005, the green pigments coded in the Color Index under the references CI 61565 61570, 74260, the orange pigments coded in the Color Index as CI11725, 15510, 45370, 71105, the red pigments coded in the Color Index as CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indole or phenolic derivatives as described in patent FR2679 771.

These pigments can also be in the form of composite pigments such as described in patent EP1184426. These composite pigments can be composed in particular of particles comprising an inorganic core covered at least partially with an organic pigment and at least one binder ensuring the fixing of the organic pigments on the core.

The pigment can also be a lacquer. The term “lacquer” is used to mean insolubilized dyes adsorbed on insoluble particles; the assembly thus obtained remaining insoluble during use.

Examples of inorganic substrates on which dyes are adsorbed are alumina, silica, calcium sodium borosilicate or calcium aluminum borosilicate, and aluminum.

Organic dyes include cochincal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090).

As examples of lacquers, mention may be made of the product known under the name D&C Red 7 (CI 15 850:1).

The pearlescent materials can be chosen from white pearlescent pigments such as natural or synthetic mica (fluorphlogopite), mica coated with titanium or bismuth oxychloride, colored pearlescent pigments such as titanium mica with iron oxides, titanium mica with, in particular, ferric blue or chromium oxide, titanium mica with an organic pigment of the above-mentioned type, as well as pearlescent pigments based on bismuth oxychloride.

Preferably, the composition according to the present invention comprises one or more pigment of the mineral type, in particular chosen from metal oxides, and more particularly chosen from titanium dioxides, iron oxides, more preferably chosen from titanium dioxides comprising a lipophilic or hydrophobic coating, iron oxides comprising a lipophilic or hydrophobic coating, and their mixtures,

More particularly the composition according to the present invention comprises one or more pigment chosen titanium from dioxides, iron oxides and their mixtures, the said metallic oxides being coated with an N-acylated amino acid derivative, in particular a glutamic acid derivative and/or a salt thereof, and more particularly a stearoyl glutamate, such as aluminum stearoyl glutamate.

In an embodiment of the present invention, the pigment is preferably selected from titanium dioxide anatase coated with aluminium stearoyl glutamate (hydrophobic), yellow iron oxide treated with disodium stearoyl glutamate (hydrophobic), red iron oxide treated with disodium stearoyl glutamate (hydrophobic), black iron oxide treated with disodium stearoyl glutamate (hydrophobic), or mixtures thereof.

The amount of pigments present in the composition, according to embodiments herein, may vary. The pigment, according to embodiments herein, may be present in the composition in a weight range of about 10% to about 40%, preferably in a weight range of about 20% to about 30% relative to the total weight of the composition.

For example, in various embodiments, the amount of pigments may range from about 10% to about 40%, from about 10% to about 35%, from about 10% to about 30%, from about 10% to about 25%, from about 12% to about 40%, from about 12% to about 35%, from about 12% to about 30%, from about 12% to about 25%, from about 15% to about 40%, from about 15% to about 35%, from about 15% to about 30%, from about 15% to about 25% from about 18% to about 40%, from about 18% to about 35%, from about 18% to about 30%, from about 18% to about 25% from about 20% to about 40%, from about 20% to about 35%, from about 20% to about 30%, or from about 20% to about 25% relative to the total weight of the composition.

For example, in various embodiments, the amount of pigments may be about 10%, about 12%, about 15%, about 18%, about 20%, about 25%, about 30%, about 35% or about 40%, by weight, relative to the total weight of the composition, including any range using any of the foregoing as upper and lower limits.

In the present invention, the cosmetic composition is substantially free of silicones and alkanes.

“Silicones” as it is used herein means silicone-containing components that include volatile silicone oils, non-volatile silicone oils, silicone-based surfactants, and metal oxide pigments that have been treated with silicone-based surface treatment agents. The term “silicones” is used herein to encompass any compound consisting of siloxane (—R2Si—O—SiR2—, where R=organic group) linkage, with two organic groups, attached to each silicon atom

“Alkanes” as it is used herein means alkane-based volatile oil which is usually liquid in an environment at ordinary temperature and pressure (for example, 250° C., 1 atmosphere). The alkane-based volatile oils are linear or branched alkanes of 8 to 19 carbon atoms. By “linear” it is meant carbons are bounded together in a straight chain like structure. By “branched” it is meant there are one or more branches of alkyl group on the straight chain. Example of which include nonane, decane, undecane, dodecane, tridecane, tetradecane, pentadecane, hexadecane, heptadecane, octadecane, nonadecane, and their structural isomers (such as isododecane or isohexadecane) and mixture thereof.

“Substantially free” as it is used herein means that while it is preferred that no amount of the specific component be present in the composition, it is possible to have small amounts of it in the compositions of the invention provided that these amounts do not materially affect at least one, preferably most, of the advantageous properties of the compositions of the invention. In certain embodiments, substantially free means less than about 2% of the identified ingredient, such as less than about 1%, such as less than about 0.5% based on the composition as a whole (or a particular component, if indicated).

### Fatty Solid Compound

The composition according to the invention may optionally comprise one or more fatty solid compound. By “fatty solid compound” it is meant a fatty substance which is solid at room temperature. In certain embodiment, the fatty solid compound (at ambient temperature and atmospheric pressure), may be chosen from waxes, pasty compounds, or mixtures thereof, more particularly from hydrocarbon-based waxes, pasty hydrocarbon-based compounds, or mixtures thereof.

### Waxes

By “wax”, within the meaning of the invention, refers to a lipophilic compound which is solid at room temperature, having a melting point greater than or equal to 40° C., and less than or equal to 120° C., more particularly less than or equal to 90° C.

Within the meaning of the invention, the melting temperature (or melting point) corresponds to the temperature of the most endothermic peak observed in thermal analysis (DSC) as described in standard ISO 11357-3; 1999.

The melting point of a solid fatty compounds can be measured using a differential scanning calorimeter (DSC), for example the calorimeter sold under the name “DSC 2000” by the company TA Instruments with the software “TA Universal Analysis”.

The measurement protocol is as follows:
A sample of 5 mg of wax is placed in a crucible and subjected to an initial temperature rise ranging from −20° C. to 120° C., at a heating rate of 10° C./minute, then cooled by 120° C. at −20° C. at a cooling rate of 10° C./minute and finally subjected to a second temperature rise ranging from −20° C. to 120° C. at a heating rate of 5° C./minute. During the second rise in temperature, the melting point of the solid fatty substance is measured, corresponding to the temperature of the most endothermic peak of the melting curve observed.

More particularly, the wax can be chosen from polar waxes, in particular alcohol waxes; esters such as waxes of animal or vegetable origin, such as beeswax, lanolin, sunflower, candelilla, carnauba, rice bran, montan, hydrogenated jojoba oil; waxes resulting from the hydrogenation of esters obtained from C6-C22 fatty alcohols of vegetable origin and vegetable oil; waxes of formula R1COOR2 in which R1 and R2 represent linear, branched or cyclic aliphatic chains whose number of atoms varies from 10 to 50, which can contain a heteroatom in particular oxygen, and whose melting temperature varies from 40 at 120° C.; partial or total, preferably total, esters of a saturated C16-C30 carboxylic acid with glycerol, such as glyceryl tristearate or tribehenate, glyceryl trihydroxystearate, as well as mixtures thereof.

Mention may also be made of apolar hydrocarbon waxes (in other words made up solely of carbon and hydrogen atoms), resulting from the transformation of petroleum, such as for example paraffins, microcrystalline waxes, ozokerites, polyethylene waxes, polymethylene waxes (Synthetic Wax, Fischer-Tropsch), alone or in mixtures.

### Pasty Compounds

By “pasty compound”, within the meaning of the present invention, refers to a lipophilic compound comprising, at an ambient temperature, a liquid fraction, and a solid fraction. Thus, a pasty compound may have a starting melting point below 25° C.

The melting point of the pasty compound is determined according to the same principle as that detailed above for the waxes. In case of a pasty compound, the measurement protocol is however as follows:
A sample of 5 mg of pasty compound placed in a crucible is subjected to a first rise in temperature ranging from −20° C. to 100° C., at a heating rate of 10° C./minute, then is cooled by 100° C. to −20° C. at a cooling rate of 10° C./minute and finally subjected to a second temperature rise ranging from −20° C. to 100° C. at a heating rate of 5° C./minute. The melting point of the pasty compound is the temperature value corresponding to the apex of the peak of the curve representing the variation of the difference in power absorbed as a function of temperature.

It should be noted that the liquid fraction by weight of the pasty compound at ambient temperature is equal to the ratio of the enthalpy of fusion consumed at ambient temperature to the enthalpy of fusion of the pasty fatty substance. The enthalpy of fusion of the pasty compound is the enthalpy consumed by the latter to pass from the solid state to the liquid state. The pasty compound is said to be in the solid state when all of its mass is in crystalline solid form. The pasty compound is said to be in the liquid state when all of its mass is in liquid form. The enthalpy of fusion of the pasty compound is the quantity of energy necessary to change the pasty compound from the solid state to the liquid state. It is expressed in J/g. The enthalpy of fusion of the pasty fatty substance is equal to the area under the curve of the thermogram obtained.

Preferably, the pasty compounds also called as pasty hydrocarbon-based compounds are chosen from petroleum jelly; vegetable butters, such as in particular mango, shea, cupuacu, murumuru, cocoa, babassu or jojoba butters; totally or partially hydrogenated vegetable oils, such as for example hydrogenated soybean oil, hydrogenated copra oil, hydrogenated rapeseed oil, mixtures of hydrogenated vegetable oils such as products with the INCI name hydrogenated vegetable oil, partially hydrogenated olive oil; esters of hydrogenated castor oil and C16-C22 fatty acids, bis-diglyceryl polyacyladipate-2; products with the INCI name hydrogenated coco-glycerides; dilinoleic alcohol and/or dilinoleic acid dimer esters, for example the products with the following INCI names: bis-behenyl/isostearyl/phytosteryl dimer dilinoleyl dimer dilinoleate, phytosteryl/isostearyl/cetyl/stearyl/behenyl dimer dilinoleate), hydrogenated castor oil dimer dilinoleate, or mixtures thereof.

According to a preferred embodiment of the present invention, the amount of fatty compound, may be present in the composition in a weight range of about of about 0.05% to about 4%, such as about 0.2% to about 3.5%, about 0.2% to about 3%, about 0.2% to about 2.5%, about 0.2% to about 2%, about 0.2% to about 1.5%, about 0.4% to about 3.5%, about 0.4% to about 3%, about 0.4% to about 2.5%, about 0.4% to about 2%, about 0.4% to about 1.5%, about 0.6% to about 3.5%, about 0.6% to about 3%, about 0.6% to about 2.5%, about 0.6% to about 2%, about 0.6% to about 1.5%, about 0.8% to about 3.5%, about 0.8% to about 3%, about 0.8% to about 2.5%, about 0.8% to about 2%, about 0.8% to about 1.5%, about 1% to about 3.5%, about 1% to about 3%, about 1% to about 2.5%, about 1% to about 2%, or about 1% to about 1.5% relative to the total weight of the composition.

In some embodiments, fatty compound in the composition may be about 0.2%, about 0.4%, about 0.6%, about 0.8%, about 1%, about 1.5%, about 2%, about 2.5%, about 3% or about 3.5% by weight, relative to the total weight of the composition, including any range using any of the foregoing as upper and lower limits.

### Other Ingredients

he composition, according to embodiments herein, may also include one or more additional components, particularly cosmetic ingredients, which may be notably chosen from solvents, salts, preservatives, vitamins, extracts, skincare active agents, chelating agents, brighteners, antioxidants, fragrances, neutralizers, UV absorbers, humectants, and mixtures thereof. In some embodiments, the compositions are free or essentially free of any of the aforementioned.

### Solvents

The composition, according to embodiments herein, may include one or more solvents, which are used to facilitate the deposition of ingredients of the composition on the skin.

According to embodiments herein, the solvent is a volatile, water-soluble solvent and is part of the aqueous phase, of the composition.

The term “aqueous phase” is used to mean a phase containing water and optionally other water-soluble or water miscible ingredients.

The aqueous phase of the composition in the present invention may include water. In various embodiments, water may be present in a weight range of about 10% to about 60%, such as about 15% to about 55%, about 15% to about 50%, about 15% to about 45%, about 15% to about 40%, about 20% to about 55%, about 20% to about 50%, about 20% to about 45%, about 20% to about 40%, about 25% to about 55%, about 25% to about 50%, about 25% to about 45%, about 25% to about 40%, about 30% to about 55%, about 30% to about 50%, about 30% to about 45%, or about 30% to about 40% relative to the total weight of the composition.

In some embodiments, water in the composition may be about 15%, about 20%, about 25%, about 30%, about 35%, about 40%, about 45%, about 50% or about 55% by weight, relative to the total weight of the composition, including any range using any of the foregoing as upper and lower limits.

The term “water-soluble solvent” used herein refers to a compound, which is liquid at ambient temperature and water-miscible (miscibility in water of greater than 50% by weight at 25° C. and atmospheric pressure).

Examples of water-soluble solvents that can be suitably used in the composition in accordance with the present invention include, lower monoalcohols having 1 to 5 carbon atoms, such as ethanol and isopropanol, glycols having 2 to 8 carbon atoms, such as ethylene glycol, propylene glycol, 1,3-butylene glycol, propanediol, pentylene glycol, glycerol and dipropylene glycol, C3-C4 ketones and C2-C4 aldehydes.

The amount of water-soluble solvents present in the composition, according to embodiments herein, may vary. The water-soluble solvents, according to embodiments herein, may be present in the composition in a weight range of about 2% to about 22%, such as about 5% to about 20%, of about 5% to about 19%, of about 5% to about 18%, about 5% to about 17%, about 5% to about 16%, about 5% to about 15%, about 5% to about 14%, about 5% to about 13%, about 8% to about 20%, of about 8% to about 19%, of about 8% to about 18%, about 8% to about 17%, about 8% to about 16%, about 8% to about 15%, about 8% to about 14%, about 8% to about 13%, about 10% to about 20%, of about 10% to about 19%, of about 10% to about 18%, about 10% to about 17%, about 10% to about 16%, about 10% to about 15%, about 10% to about 14% or about 10% to about 13% relative to the total weight of the composition.

In some embodiments, water-soluble solvents in the composition may be about 5%, about 8%, about 10%, about 12%, about 14%, about 16%, about 18% or about 20%, by weight, relative to the total weight of the composition, including any range using any of the foregoing as upper and lower limits.

### Salts

The composition, according to embodiments herein, may include one or more salts, which are used to stabilize the water-in-oil emulsion.

According to embodiments herein, the salt is inorganic in nature and is part of the aqueous phase of the composition.

Examples of salts include but are not limited to magnesium sulphate, sodium chloride, potassium chloride, or mixtures thereof.

The amount of salts present in the composition, according to embodiments herein, may vary. The salts, according to embodiments herein, may be present in the composition in a weight range of about 0.05% to about 3.5%, such as from about 0.1% to about 3%, about 0.1% to about 2.5%, about 0.1% to about 2%, about 0.1% to about 1.5%, about 0.1% to about 1%, about 0.2% to about 3%, about 0.2% to about 2.5%, about 0.2% to about 2%, about 0.2% to about 1.5%, about 0.2% to about 1%, about 0.3% to about 3%, about 0.3% to about 2.5%, about 0.3% to about 2%, about 0.3% to about 1.5%, about 0.3% to about 1%, about 0.4% to about 3%, about 0.4% to about 2.5%, about 0.4% to about 2%, about 0.4% to about 1.5%, or about 0.4% to about 1% relative to the total weight of the composition.

In some embodiments, salts present in the composition may be about 0.1%, about 0.2%, about 0.25%, about 0.3%, about 0.4%, about 1%, about 1.5%, about 2%, about 2.5% or about 3%, by weight, relative to the total weight of the composition, including any range using any of the foregoing as upper and lower limits.

### Preservatives

The composition, according to embodiments herein, may optionally include one or more preservatives, which are used to preserve the integrity and stability of cosmetic products that can be damaged by microorganisms. Preservatives are essential to guarantee consumer's safety and to increase the shelf-life of the cosmetic product.

Examples of preservatives include but are not limited to phenoxyethanol, chlorophenesin, or mixtures thereof. Non-limiting examples of preservatives include benzyl alcohol, parabens (methylparaben, ethylparaben, propylparaben, butylparaben, isobutylparaben, etc.), sodium benzoate, salicylic n sorbate, ethylhexylglycerin, DMDM Hydantoin, Formaldahyde, Triclosan, Chlorhexidine digluconate, Imidazolidinyl Urea, and Diazolidinyl Urea or combinations thereof.

The amount of preservatives present in the composition, according to embodiments herein, may vary. The preservatives, according to embodiments herein, may be present in the composition in a weight range of about 0.05% to about 4%, such as from about 0.2% to about 3.5%, about 0.2% to about 3%, about 0.2% to about 2.5%, about 0.2% to about 2%, about 0.2% to about 1.5%, about 0.4% to about 3.5%, about 0.4% to about 3%, about 0.4% to about 2.5%, about 0.4% to about 2%, about 0.4% to about 1.5%, about 0.6% to about 3.5%, about 0.6% to about 3%, about 0.6% to about 2.5%, about 0.6% to about 2%, about 0.6% to about 1.5%, about 0.8% to about 3.5%, about 0.8% to about 3%, about 0.8% to about 2.5%, about 0.8% to about 2%, about 0.8% to about 1.5%, about 1% to about 3.5%, about 1% to about 3%, about 1% to about 2.5%, about 1% to about 2%, or about 1% to about 1.5% relative to the total weight of the composition.

In some embodiments, preservatives present in the composition may be about 0.2%, about 0.4%, about 0.6%, about 0.8%, about 1%, about 1.5%, about 2%, about 2.5%. about 3% or about 3.5%, by weight, relative to the total weight of the composition, including any range using any of the foregoing as upper and lower limits.

### Vitamins and Extracts

The composition according to embodiments herein, may optionally include one or more vitamins. In some embodiments, the composition may comprise a plurality of vitamins. Such vitamins include materials having vitamin activity. In some embodiments, the vitamins may be present in a total amount no more than about 2% by weight of the composition. In some embodiments, the vitamins may be present in a total amount no more than about 1.5% by weight of the composition. In some embodiments, the vitamins may be present in a total amount no more than about 1% by weight of the composition. In some embodiments, the vitamins may be present in a total amount no more than about 0.5% by weight of the composition. In some embodiments, the vitamins may be present in a total amount no less than about 0.04% by weight of the composition. In some embodiments, the vitamins may be present in a total amount of about 0.01% to about 0.08% by weight of the composition, such as about 0.02% to about 0.06%, about 0.02% to about 0.05%, about 0.02% to about 0.04%, about 0.03% to about 0.06% about 0.03% to about 0.05% or about 0.03% to about 0.04% relative to the total weight of the composition.

Non-limiting examples of such vitamins include Vitamin K, Vitamin B8, Vitamin B12, Thiamine riboflavin, Nicotinamide (also referred to as Niacinamide), Pantothenic acid, Pyridoxine and derivatives, biotin (vitamin B7), folic acid, cyanocobalamin and Ascorbic acid. Vitamins include vitamin derivatives. For example, ascorbic acid (vitamin C) and derivatives thereof, especially the phosphate derivatives thereof such as the potassium salt of di-alpha-tocopheryl diascorbyl phosphate (sold by the company Senju Pharmaceutical under the reference Sepivital EPC), magnesium ascorbyl phosphate and sodium ascorbyl phosphate (sold by the company Roche under the reference Stay-C 50), and esters thereof such as ascorbyl acetate, palmitate and propionate; retinol (vitamin A) and derivatives thereof, especially esters thereof such as retinyl acetate, palmitate and propionate; as well as pantothenic acid (vitamin B) and derivatives thereof such as pantolactone, and D-panthenol.

The composition according to embodiments herein, may optionally include one or more extract. In some embodiments, the composition may include a single extract. In some embodiments, the composition may include a plurality of extracts. In some embodiments, the extract may be present in a total amount no more than about 1.5% by weight of the composition. In some embodiments, the extract may be present in a total amount no more than about 1% by weight of the composition. In some embodiments, the vitamins may be present in a total amount no more than about 0.5% by weight of the composition. In some embodiments, the vitamins may be present in a total amount no more than 0.25% by weight of the composition. In some embodiments, the vitamins may be present in a total amount no less than 0.001% by weight of the composition. Non-limiting examples of extracts include, e.g., plant extracts.

### Composition Preparation

The compositions according to the invention may be manufactured using known processes, generally used in the cosmetics field. Typically, the process may include blending the ingredients in phase A into the main kettle at 80-85° C. temperature to obtain a homogeneous uniform melted mixture. In a separate container ingredient in phase B may be blended at 80-85° C. temperature to obtain a homogeneous uniform mixture. Slowly phase B may be added to phase A to form homogeneous emulsion. Optionally, one or more fragrances, antioxidants, humectants, or mixtures thereof may suitably be added. The emulsion may be cooled down at 25° C. and phase C may be added to emulsion to obtain uniform smooth homogenous composition.

### Method of Use

The composition, according to embodiments herein, may more particularly be a composition for making up the skin. Accordingly, the composition, in various embodiments herein, may be in the form of a makeup base composition. Preferably, the composition is in the form of a primer, foundation, or concealer, more preferably a liquid concealer.

The composition, according to embodiments herein, can be applied using any means, such as finger or preferably using an applicator, for example using a dip applicator, flocked dip applicator, brushes and/or even using a sponge.

Accordingly, embodiments herein include a method for for making up the skin, comprising at least one step of applying the composition as described herein, to skin.

Although the present invention has been described in considerable detail with reference to certain embodiments and implementations thereof, other embodiments are possible to cover the modifications and variations of the present invention.

### EXAMPLES

The invention will now be illustrated with examples, which is intended to illustrate the invention without limiting the scope of the present invention. Ingredient amounts are indicated in the following examples in active material weight percentages, relative to the total weight of the composition.

### Inventive Examples

The compositions below in Table 1 (Inventive Examples), were made by first mixing the oils, oil-soluble ingredients in a container, mixing the water and water-soluble ingredients in a separate container, then while homogenizing, combining the two phases, and then adding the colorants. The inventive examples are substantially free of silicones and alkanes.

TABLE 1
(Inventive Examples)

| | Inventive Examples | | |
|---|---|---|---|
| | IE1 | IE2 | IE3 |
| | Content (% By Weight Relative to The Total Weight of The | | |
| Ingredients | Composition) | | |
| Ethylhexyl palmitate | 11.67 | 11.67 | 11.67 |
| Hydrogenated | 9.72 | 9.72 | 9.72 |
| polyisobutene | | | |
| Sortbitan isostearate | 4.87 | 4.86 | 7 |
| Silica silylate | 0.39 | 1 | 0.39 |
| Disteardimonium | 0.58 | 0.58 | 0.58 |
| hectorite | | | |
| Pigments | 22.49 | 22.49 | 22.49 |
| Solid fatty compounds | 1.67 | 1.67 | 1.67 |
| Preservatives | 1.05 | 1.05 | 1.05 |
| Solvents (including water) | 46.51 | 46.51 | 46.51 |
| Other ingredients | QS | QS | QS |

Certain characteristics such as stability, texture, coverage, uniform film formation, finish and overall performance of the compositions were evaluated and compared, the summary of which is shown in Table 2. below.

TABLE 2
(Summary of Characteristics)

| | Inventive Examples | | |
|---|---|---|---|
| Characteristics | IE1 | IE2 | IE3 |
| Stability | | | |
| 25° C. | Stable | Stable | Stable |
| 45° C. | Stable | Stable | Stable |
| Texture | Smooth feel | Some feel of | Some feel of |
| | | dragginess and | dragginess and |
| | | thickness | stickiness |
| Coverage | Medium to | Medium to high | Medium to high |
| | high | | |
| Uniform film | Even film | Even film | Even film |
| Finish | Natural to | Natural to radiant | Natural to radiant |
| | radiant | | |
| Overall performance | After | After application, | After application, |
| | application, | Light weight, | some feeling of |
| | Light weight, | smooth creamy | heaviness and |
| | smooth | feel, conceal with | tack. Conceal with |
| | creamy, | flawless natural to | natural to radiant |
| | conceal with | matte finish. | finish. |
| | flawless natural | | |
| | to radiant | | |
| | finish. | | |

### Comparative Examples

The compositions below in Table 3 (Comparative Examples), were made by first mixing the oils, oil-soluble ingredients in a container, mixing the water and water-soluble ingredients in a separate container, then while homogenizing, combining the two phases, and then adding the colorants.

TABLE 3
(Comparative Examples)

| | Comparative Examples | | | | |
|---|---|---|---|---|---|
| | CE1 | CE2 | CE3 | CE4 | CE5 |
| Ingredients | Content (% By Weight Relative to The Total Weight of The Composition) | | | | |
| Ethylhexyl palmitate | 0 | 0 | 11.67 | 11.67 | 11.67 |
| Isononyl isononanoate | 0 | 11.67 | 0 | 0 | 0 |
| Coco-caprylate/caprate | 11.66 | 0 | 0 | 0 | 0 |
| Hydrogenated | 9.72 | 9.72 | 9.72 | 9.72 | 9.72 |
| polyisobutene | | | | | |
| Sortbitan isostearate | 4.86 | 4.86 | 4.86 | 1 | 2.43 |
| Polyglyceryl-6- | 0 | 0 | 0 | 0 | 2.43 |
| polyricinoleate | | | | | |
| Silica silylate | 0.39 | 0.39 | 0.00 | 0.39 | 0.39 |
| Disteardimonium hectorite | 0.60 | 0.60 | 0.58 | 0.58 | 0.58 |
| Pigments | 22.49 | 22.49 | 22.49 | 22.49 | 22.49 |
| Solid fatty compounds | 1.17 | 1.17 | 1.67 | 1.67 | 1.67 |
| Preservatives | 1.18 | 1.18 | 1.05 | 1.05 | 1.05 |
| Solvents (including water) | 46.50 | 46.50 | 46.51 | 46.51 | 46.51 |
| Other ingredients | QS | QS | QS | QS | QS |

Certain characteristics such as stability. texture. coverage, uniform film formation, finish and overall performance of the compositions were evaluated and compared, the summary of which is shown in Table 4. below.

TABLE 4
(Summary of Characteristics)

| | Comparative Examples | | | | |
|---|---|---|---|---|---|
| Characteristics | CE1 | CE2 | CE3 | CE4 | CE5 |
| Stability | | | | | |
| 25° C. | Unstable | Unstable | Unstable | Unstable | Unstable |
| 45° C. | Unstable | Unstable | Unstable | Unstable | Unstable |
| Texture | Very greasy | Very greasy | Greasy feel | Very slippery | Very |
| | feel | feel | | feel | greasy |
| | | | | | feel |
| Coverage | Medium to | Medium to | Medium to full | Medium to | Medium to |
| | full | full | | full | full |
| Uniform film | Even film | Even film | Even film | Not even | Even film |
| Finish | Natural to | Natural to | Natural to | Natural to | Natural to |
| | radiant | radiant | radiant | radiant | radiant |
| Overall | Light | Light | Light weight, | Light weight, | Light weight, |
| performance | weight, | weight, | shiny and | shiny/greasier | shiny/greasier |
| | shiny and | shiny and | greasier feel | feel | feel |
| | greasier | greasier feel | | | |

### Result

As observed from the Table 2 & Table 4 above, the inventive examples (IE1, IE2& IE3) were surprisingly stable. whereas the comparative examples (CE1, CE2, CE3, CE4 & CE5) all are unstable.

What is claimed is:

1. A composition in the form of a water-in-oil emulsion, comprising:
(a) one or more esters formed from at least one fatty acid having more than 13 carbon atoms;
(b) a polymer component comprising one or more polymerized (iso)butenes;
(c) at least about 3% by weight of one or more carbohydrate fatty acid ester emulsifiers;
(d) one or more fillers comprising hydrophobic silica; and
(e) one or more pigments, wherein the composition is substantially free of silicones and alkanes.

2. The composition of claim 1, wherein the one or more esters formed from at least one fatty acid having more than 13 carbon atoms is formed from one or more fatty acids selected from myristic acid, palmitic acid, stearic acid, arachidic acid, behenic acid, myristoleic acid, palmitoleic acid, sapienic acid, oleic acid linoleic acid, and combinations thereof.

3. The composition of claim 1, wherein the one or more esters formed from at least one fatty acid having more than 13 carbon atoms is selected from a group consisting of esters of myristic acid, esters of palmitic acid, esters of stearic acid and mixtures thereof.

4. The composition of claim 1, wherein the one or more esters formed from at least one fatty acid having more than 13 carbon atoms is present in the composition in an amount ranging from about 8% to about 15% by weight relative to the total weight of the composition.

5. The composition of claim 1, wherein the one or more polymerized (iso)butenes is selected from a group consisting of polyisobutene, hydrogenated polyisobutene, and mixtures thereof.

6. The composition of claim 1, wherein the polymer component comprising polymerized (iso)butene is a hydrogenated polyisobutene.

7. The composition of claim 1, wherein the one or more polymerized (iso)butenes is hydrogenated polyisobutene and is present in the cosmetic composition in an amount ranging from about 8% to about 12% by weight relative to the total weight of the cosmetic composition.

8. The composition of claim 1, wherein the one or more carbohydrate fatty acid ester emulsifiers is selected from sugar fatty acid esters comprising one sugar alcohol and one fatty acid.

9. The composition of claim 8, wherein the sugar alcohol is selected from a group consisting of mannitol, erythritol, xylitol, sorbitol, arabitol, pentaerythritol, and mixtures thereof.

10. The composition of claim 8, wherein the fatty acid is selected from C₈-C₂₄ fatty acids and mixtures thereof.

11. The composition of claim 1, wherein the one or more carbohydrate fatty acid ester emulsifiers is selected from a group consisting of sorbitan palmitate, sorbitan isostearate, sorbitan trioleate and mixtures thereof.

12. The composition of claim 1, wherein the one or more carbohydrate fatty acid ester emulsifier is sorbitan isostearate and is present in the cosmetic composition in an amount ranging from about 3% to about 7% by weight relative to the total weight of the cosmetic composition.

13. The composition of claim 1, wherein the one or more fillers comprising hydrophobic silica is selected from a group consisting of hydrophobically treated pyrogenic silica, hydrophobic silica aerogel, and mixtures thereof and is present in the composition in an amount ranging from about 0.1% to about 1% by weight relative to the total weight of the composition.

14. The composition of claim 1, wherein the one or more fillers further comprises clay and wherein the clay is selected from a group consisting of hydrophobic hectorite, hydrophobic bentonite, and mixtures thereof.

15. The composition of claim 1, wherein the one or more fillers comprising hydrophobic silica and the polymer component comprising polymerized (iso)butene are present in a weight ratio of hydrophobic silica to polymerized (iso)butene that is from about 1:50 to about 1:10.

16. The composition of claim 1, wherein the one or more pigments are selected from mineral pigments, organic pigments, pearlescent pigments, and mixtures thereof.

17. The composition of claim 1, wherein the composition further comprises one or more fatty solid compounds selected from waxes, pasty compounds, or mixtures thereof.

18. The composition of claim 1, wherein the composition further comprises one or more other ingredients selected from solvents, salts, preservatives, vitamins, extracts, skincare active agents, chelating agents, brighteners, antioxidants, fragrances, neutralizers, UV absorbers, humectants, and mixtures thereof.

19. A method for making up the skin, characterized in that said method comprises topically applying said composition as claimed in claim 1 in order to conceal imperfections of the skin.
